(19) United States
(12) Patent Application Publication
(10) Pub. No.: US 2003/0206897 A1
(43) Pub. Date: Nov. 6, 2003

(54) Cosmetic compositions

(75) Inventors: Conor James O'Prey, Richmond (GB); Marina Trani, Ascot (GB); David John Weisgerber, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/388,017

(22) Filed: Mar. 12, 2003

Related U.S. Application Data
(63) Continuation of application No. PCT/US00/25086, filed on Sep. 13, 2000.

(51) Int. Cl. A61K 38/48

(52) U.S. Cl. 424/94.64

(57) ABSTRACT

An oil in water emulsion are useful for treating skin or hair comprising:

(a) from about 0.0001% to about 1% protease enzyme;

(b) less than 20% polyhydric alcohol in the continuous phase;

(c) less than 50% oil phase; is disclosed;

wherein the protease enzyme exhibits its activity at pH greater than 5.5 and wherein the water activity of the external phase is greater than 0.85. In a preferred embodiment the composition is packaged in a pack comprising two or more chambers.

### CROSS REFERENCE TO RELATED APPLICATION(S)

This application is a continuation of International Application No. PCT/US00/25086, filed Sep. 13, 2000.

### FIELD OF THE INVENTION

The present invention relates to topical oil in water emulsion compositions which comprise a protease enzyme, less than 20% polyhydric alcohol in the continuous phase and less than 50% oil phase. More particularly this invention relates to oil in water emulsions which are suitable for providing high levels of desquamation/skin exfoliation such that use of the product leads to noticeable improvement in skin condition and which can significantly slow, and even reverse, the signs of ageing. Furthermore this invention relates to the stabilisation of the protease enzyme activity within such oil in water emulsion compositions via the use of a dual chamber dispensing pack. The compositions of the present invention are especially useful as leave on products for the treatment of skin and/or hair.

### BACKGROUND

Skin is made up of several layers of cells which coat and protect the keratin and collagen fibrous proteins that form the skeleton of its structure. The outermost of these layers, referred to as the stratum corneum, is known to be composed of 25 nm protein bundles surrounded by 8 nm thick layers. Anionic surfactants and organic solvents typically penetrate the stratum corneum membrane and, by delipidization (i.e. removal of the lipids from the stratum corneum), destroy its integrity. This destruction of the skin surface topography leads to a rough feel and may eventually permit the surfactant or solvent to interact with the keratin, creating irritation.

Dry, itchy or flaky skin may also result from the failure to maintain a proper water gradient across the stratum corneum. Most of the water needed to maintain the water gradient, which is sometimes considered to be the stratum corneum's plasticizer, comes from inside the body. If the humidity is too low, such as in a cold climate, insufficient water remains in the outer layers of the stratum corneum to properly plasticize the tissue, and the skin begins to scale and becomes itchy.

Recently, across many fields, there has been an increase in the use of biologically active enzymes as ingredients in a wide range of different types of consumer products. This increase has been especially prominent in the cosmetic field, in particular in leave on topical compositions for the skin or hair, where these biologically active enzymes have the benefit of being highly efficacious when compared with other more traditional ingredients. Furthermore, in some cases, the use of biologically active enzymes is the only way to achieve certain cosmetic benefits, there being no equivalent traditional ingredient.

Various different enzymes are currently of particular interest as key biological actives in cosmetic compositions. Examples of such enzymes include proteases which can support or replace α-hydroxy acids in skin peeling preparations (JP-A-04027388) or which can provide desquamatory action for treatment of dry, itchy or flaky skin (EP-A-0710478); glutathion sulphydryl oxidase which can be used in hair-wave setting (JP-A-4005220); glycosidases which can enhance the process of skin desquamation (WO93/19731) and transglutaminase which can aid the formation of a protective layer on skin, hair and nails (WO94/18945, JP-A-02204407).

On of the key issues to date with using enzymes, particularly protease enzymes, in cosmetic compositions, particularly oil in water emulsions, has been the inadequate stability of the enzymes themselves within the final composition. In the case of protease enzymes the main driver of low enzyme stability is the availability of water in the composition which causes the enzyme to autolyse. The result is a reduction in the activity of the enzyme within the product and as such the final composition may not deliver the desired benefits. In addition, another key issue with the use of enzymes within cosmetic compositions is that there is a potential that side effects, such as allergic reactions or sensitisation and the like, to occur if the consumer is exposed to excessively high doses of the enzyme material. As such it is important to minimise the risk by formulating enzyme comprising compositions to comprise the minimum level of enzyme to deliver the desired end benefit. Net, excellent stability of the enzyme within the formulation has even further importance such that compositions can be formulated with low enzyme level but still deliver the desired benefit when used.

Although the prior art does not deal explicitly with addressing the potential safety issues of use of enzymes within these compositions, several innovative approaches have been taken to ensuring that the enzyme remains stable within the final composition. Approaches disclosed in the art include encapsulating the enzymes prior to inclusion within the cosmetic composition (GB 1,255,284 and JP 10-251122); buffering the cosmetic composition such that the enzyme remains inactive until used (WO 97/47238); and using precursors or other actives within the composition to stabilise the enzyme (EP 0710478 and SU 1690764). However the favoured approach in the art for stabilising protease enzymes in cosmetic compositions is to dramatically reduce water availability within the composition such that the enzyme does not autolyse. Water availability within the composition can be measured as water activity. In general the water activity of the compositions is reduced by formulating the aqueous phase of any composition with very high levels of polyhydric alcohol such as glycerine, disclosed in JP 1283213, JP 3294211. Unfortunately such systems have unacceptable aesthetics for cosmetic products. This has been overcome to date by formulation of the aqueous phase into a water in oil emulsion (U.S. Pat. No. 5,932,234 and U.S. Pat. No. 5,830,449) or into a triple water in oil in water emulsion (EP 0779071). A further solution has been to two store the product as a dual phase system within a single pack wherein the first phase comprises the stabilised enzyme in high levels of polyhydric alcohol and the second phase comprises an aqueous cosmetic composition such that when the two phases are dispensed and mixed the final aqueous composition has acceptable aesthetics (WO 97/27841).

In general the most common type of cosmetic compositions, particularly leave on compositions, are oil in water emulsion systems. In preparing such compositions one skilled in the art has a wide range of formulation possibilities including being able to balance high levels of emollients and other moisturising actives with highly preferred consumer aesthetics of a non sticky/tacky good skin feel with being fast to apply or dry on the skin. As such it would be useful to be able to develop a stable oil in water emulsion comprising protease enzyme able to deliver to the consumer good desquamation/exfoliation benefits whilst simultaneously keeping the level of enzyme as low as possible to minimise consumer risk of sensitisation. Furthermore it would be useful to be able to develop such an oil in water emulsion system which also comprises humectant, emollient and other moisturising actives such that the product chassis supports the desquamation/exfoliating benefits of the enzyme by providing further moisturisation/skin hydration benefits. Finally it would be useful to be able to develop such a system which had the highly preferred consumer aesthetic attributes of non sticky/tacky and being fast drying and thus quick to apply. Whilst the prior art provides useful advances in stabilising enzymes, particularly protease enzymes, within single phase and water in oil cosmetic compositions it does not sufficiently teach how to stabilise protease enzymes within oil in water emulsion compositions.

It has now been unexpectedly found that when from about 0.0001% to about 1% protease enzyme is incorporated into an oil in water emulsion comprising less than 20% polyhydric alcohol and less than 50% oil phase a cosmetic composition is achieved which delivers exceptional skin softness benefits, minimises consumer risk of sensitisation and has acceptable product aesthetics. This invention can be further enhanced by storing the product within a dual chamber dispensing system to enhance the stability of the protease enzyme. In such a system the first chamber comprises the protease enzyme stabilised in a high level of polyhydric alcohol and the second chamber comprises the remainder of the composition, in the form of a chassis oil in water emulsion, and wherein the compositions from the first and second chamber are dispensed simultaneously, preferably through a single nozzle, to give the final oil in water emulsion composition.

It is an object of this invention to develop a stable oil in water emulsion cosmetic composition comprising low level of protease enzymes which can be used for treating skin or hair and providing superior dequamation/exfoliation and skin feel benefits. It is a further object of this invention to develop compositions wherein the consumer exposure to the protease enzyme is minimised such that the risk of side effects is minimised.

Furthermore it is an object of this invention to enhance the formulation possibilities of protease enzyme comprising compositions by inclusion of the enzyme within an oil in water emulsion. Finally it is an object of the present invention to provide compositions with improve consumer aesthetics such as skin feel and product drying.

These, and other objects of the invention, will become apparent in light of the following disclosure.

### SUMMARY OF THE INVENTION

The present invention relates to an oil in water emulsion comprising:

(a) from about 0.0001% to about 1% protease enzyme;

(b) less than 20% polyhydric alcohol in the continuous phase;

(c) less than 50% oil phase; and

wherein the protease enzyme exhibits its activity at pH greater than 5.5 and wherein the water activity of the external phase is greater than 0.85.

### DETAILED DESCRIPTION

All percentages and ratios used herein are by weight of the total composition and all measurements made are at 25° C., unless otherwise designated. Unless otherwise indicated all percentages, ratios and levels of ingredients referred to herein are based on the actual amount of the ingredient, and do not include solvent, fillers or other materials which may be combined with the ingredient in commercially available products. Chain length and degrees of ethoxylation are also specified on a weight average basis.

All publications cited herein are hereby incorporated by reference in their entirety, unless otherwise indicated.

The term “enzyme” as used herein means the enzyme, wild-type or variant, either per se, or chemically modified by the conjugation of polymer moieties.

The term “protease enzyme” as used herein refers to any enzyme whose substrate is a protein.

As used herein, the term “wild-type” refers to an enzyme produced by unmutated hosts.

As used herein, the term “variant”, means an enzyme having an amino acid sequence which differs from that of the wild-type enzyme due to the genetic mutation of the host producing that enzyme.

As used herein “enzyme activity” refers to the activity of 20 μl of enzyme solution (50 ppm) when reacted with the surface of a suitable proteinaceous substrate disc of diameter 1 cm, at room temperature over a 30 minute time period. By adjusting the pH of the enzyme buffer solution, it is possible to compare the effect of pH on enzyme activity. For the enzymes for use herein suitable activity is defined as greater than 20% of reaction complete within 30 minutes, preferably greater than 50%, more preferably greater than 75%. By using this measure it is defined that enzyme buffers of less than pH 5.5 are not suitable for use with this enzyme.

As used herein, the term “placebo” refers to a composition, including cosmetic compositions, which are essentially free from a biologically active enzyme wherein “free from” means that the biologically active enzyme is absent or present at such a low level that it does not deliver the desired end benefit. An example of such a low level would be that the final composition comprises less than 0.0001%, by weight, of a biologically active enzyme. However placebo compositions as defined herein may comprise many actives suitable for use on the skin and or hair including those which are known to those skilled in the art including skin care actives such as vitamin complexes, humectants, skin conditioning agents, sun screens and the like. Net the placebo composition can preferably be formulated such that it is designed to maintain the benefit of the biologically active enzyme throughout phase two of the treatment cycle.

The term “skin conditioning agent”, as used herein means a material, excluding those materials which are defined as biologically active, which is capable of providing a cosmetic conditioning benefit to the skin such as moisturization, humectancy (i.e. the ability to retain or hold water or moisture in the skin), emolliency, visual improvement of the skin surface and improvement in skin feel.

The term “skin hydration” as used herein refers to an improvement in skin moisture content which can be determined either by technical measures such as by use of a corneometer etc, or expert visual measures for example Fitzpatrick skin dryness scale or by consumer self assessment.

The “water activity a_{w}” of a medium containing water is the ratio of the water vapour pressure of the product “P_{H2O} product” to the vapour pressure of pure water “P_{H2O} pure” at the same temperature. It can also be expressed as the ratio of the number of molecules of water “N_{H2O}” to the total number of molecules:

“N_{H2O}+N_{dissolved substances}”, which takes account of the molecules of dissolved substances “N_{dissolved substances”,}

It is given by the following formulae:

$${a}_{w} = \frac{{}_{}^{P}{H}{}_{2}^{} {O}^{product}}{{}_{}^{P}{H}{}_{2}^{} O} = \frac{{}_{}^{N}{H}{}_{2}^{} O}{{}_{}^{N}{H}{}_{2}^{} O + {}^{N} dissolved substances}$$

Various methods can be used for measuring the water activity. The most common is the manometric method, by which the vapour pressure is measured directly.

The term “dermatologically-acceptable,” as used herein, means that the compositions, or components thereof, are suitable for use in contact with human skin without undue toxicity, incompatibility, instability, allergic response, and the like.

The term “safe and effective amount” as used herein means an amount of a compound, component, or composition sufficient to significantly induce a positive benefit, preferably a positive skin appearance or feel benefit, including independently the benefits disclosed herein, but low enough to avoid serious side effects, i.e. to provide a reasonable benefit to risk ratio, within the scope of sound medical judgement.

Active and other ingredients useful herein may be categorised or described herein by their cosmetic and/or therapeutic benefit or their postulated mode of action. However, it is to be understood that the active and other ingredients useful herein can in some instances provide more than one cosmetic and/or therapeutic benefit or operate via more than one mode of action. Therefore, classifications herein are made for the sake of convenience and are not intended to limit an ingredient to the particularly stated application or applications listed.

The elements of these compositions are described in more detail below.

Essential Components

The compositions of the present invention comprise an oil in water emulsion comprising a hydrophilic phase, especially an aqueous phase, and a hydrophobic phase e.g., a lipid, oil or oily material. As well known to one skilled in the art, the hydrophobic phase will be dispersed in the hydrophilic phase to form the emulsion. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase. The emulsion may be or comprise (e.g., in a triple or other multi-phase emulsion). The emulsion may also comprise a gel network, such as described in G. M. Eccleston, Application of Emulsion Stability Theories to Mobile and Semisolid O/W Emulsions, Cosmetics & Toiletries, Vol. 101, November 1996, pp. 73-92, incorporated herein by reference.

Oil-in-water emulsions typically comprise less than about 50% (preferably about 1% to about 30%) of the dispersed hydrophobic phase and from about 1% to about 99% (preferably from about 40% to about 90%) of the continuous hydrophilic phase. Emulsions suitable for use in the present invention comprise less than 50% oil, preferably less than 40%, more preferably from about 5% to about 25%, most preferably from about 8% to about 15%, by weight, of oil, or hydrophobic, phase. It is preferred that the oil phase components of the compositions herein comprise an emollient material or mixtures thereof, a polyol carboxylic acid ester and a silicone oil, or mixtures thereof. In preferred embodiments, the oil phase preferably comprises additional oily components such as a natural or synthetic oils selected from mineral, vegetable, and animal oils, fats and waxes, and mixtures thereof. These oily components are present in an amount of from about 0.1% to about 15%, more preferably from about 1% to about 10% by weight of composition. Preferred for use herein are for example, hydrocarbons such as mineral oils or petrolatum. Further examples suitable for use herein are disclosed in WO98/22085.

In addition it is essential that the continuos phase of compositions suitable for use herein has a water activity of greater than 0.85, preferably greater than 0.90, more preferably greater than 0.95. Furthermore it is preferred that the final composition is has a pH in the range from about 6.1 to about 10.0, preferably from about 7.0 to about 9.0, more preferably from about 8.0 to about 9.0 and even more preferably from about 8.0 to about 8.6. It is preferred that the pH of the final composition is adjusted by addition of acidic, basic or buffer salts as necessary.

Protease Enzyme

Compositions for use herein comprise from about 0.0001% to about 1%, more preferably from about 0.001% to about 0.5%, even more preferably from about 0.005% to about 0.1%, by weight, of protease enzyme. Protease enzymes suitable for use herein exhibit their activity at pH greater than about 5.5, preferably from about 6.1 to about 10.0, more preferably from about 7.0 to about 9.0, even more preferably from about 8.0 to about 9.0 and most preferably from about 8.0 to about 8.6.

Protease enzymes are classified under the Enzyme Classification number E.C. 3.4 (Carboxylic Ester Hydrolases) in accordance with the Recommendations (1992) of the International Union of Biochemistry and Molecular Biology (IUBMB). Useful proteases are also described in PCT publications: WO 95/30010 published Nov. 9, 1995 by The Procter & Gamble Company; WO 95/30011 published Nov. 9, 1995 by The Procter & Gamble Company; WO 95/29979 published Nov. 9, 1995 by The Procter & Gamble Company. Preferred protease enzymes for use herein are subtilisin, chymotrypsin and elastase-type protease enzymes.

Especially preferred for use herein are subtilisin-type protease enzymes. Subtilisin enzymes are naturally produced by Bacillus alcalophilus, Bacillus amyloliquefaciens, Bacillus amylosaccharicus, Bacillus licheniformis, Bacillus lentus and Bacillus subtilis microorganisms.

A particularly preferred substilisin-type enzyme is bacterial serine protease enzyme, and variants thereof, obtained from Bacillus amyloliquefaciens, Bacillus licheniformis and/or Bacillus subtilis, including Novo Industries A/S Alcalase®, Esperase®, Savinase® (Copenhagen, Denmark), Gist-brocades' Maxatase®, Maxacal® and Maxapem 15® (protein engineered Maxacal®) (Delft, Netherlands), and subtilisin BPN and BPN′, which are commercially available.

Especially preferred are protease enzymes, and variants thereof, obtained from Bacillus amyloliquefaciens. One known enzyme is BPN′. The wild-type BPN′ from Bacillus amyloliquefaciens is characterized by the amino acid sequence:

```
1 10 20
AlaGlnSerValProTyrGlyValSerGlnIleLysAlaProAlaLeuHisSerGlnGly
                                                      30 40
TyrThrGlySerAsnValLysValAlaValIeAspSerGlyIleAspSerSerHisPro
                                                      50 60
AspLeuLysValAlaGlyGlyAlaSerMetValProSerGluThrAsnProPheGlnAsp
                                                      70 80
AsnAsnSerHisGlyThrHisValAlaGlyThrValAlaAlaLeuAsnAsnSerIleGly
                                                      90 100
ValLeuGlyValAlaProSerAlaSerLeuTyrAlaValLysValLeuGlyAlaAspGly
                                                      110 120
SerGlyGlnTyrSerTrpIleIleAsnGlyIleGluTrpAlaIleAlaAsnAsnMetAsp
                                                      130 140
ValIleAsnMetSerLeuGlyGlyProSerGlySerAlaAlaLeuLysAlaAlaValAsp
                                                      150 160
LysAlaValAlaSerGlyValValValValAlaAlaAlaGlyAsnGluGlyThrSerGly
                                                      170 180
SerSerSerThrValGlyTyrProGlyLysTyrProSerValIIeAlaValGlyAlaVal
                                                      190 200
AspSerSerAsnGlnArgAlaSerPheSerSerValGlyProGluLeuAspValMetAla
                                                      210 220
ProGlyValSerIleGlnSerThrLeuProGlyAsnLysTyrGlyAlaTyrAsnGlyThr
                                                      230 240
SerMetAlaSerProHisValAlaGlyAlaAlaAlaLeuIleLeuSerLysHisProAsn
                                                      250 260
TrpThrAsnThrGlnValArgSerSerLeuGluAsnThrThrThrLysLeuGlyAspSer
                                                      270 275
PheTyrTyrGlyLysLysGlyLeuIleAsnAsnValGlnAlaAlaAlaGln
```

PheTyrTyrGlyLysLysGlyLeu Ile AsnAsnValGlnAlaAlaAlaGln

Variants of BPN′, hereafter referred to as “Protease A”, are disclosed in U.S. Pat. No. 5,030,378 (issued to Venegas, Jul. 9, 1991) as characterized by the BPN′ amino acid sequence with the following mutations:

a.) the Gly at position Gly166 is replaced with Asn, Ser, Lys, Arg, His, Gln, Ala or Glu; the Gly at position Gly169 is replaced with Ser; the Met at position Met222 is replaced with Gln, Phe, Cys, His, Asn, Glu, Ala or Thr; or

b.) the Gly at position Gly166 is replaced with Lys and the Met at position Met222 is replaced with Cys; or

c.) the Gly at position Gly160 is replaced with Ala and the Met at position Met222 is replaced with Ala.

Additional variants of BPN′, heretoforth referred to as “Protease B”, are disclosed by Genencor International, Inc. (San Francisco, Calif.) European Patent EP-B-251,446 (granted Dec. 28, 1994 and published Jan. 7, 1988) as characterized by the wild-type BPN′ amino acid with the mutations in one or more of the following amino acids: Tyr21, Thr22, Ser24, Asp36, Ala 45, Ala48, Ser49, Met50, His67, Ser87, Lys94, Val95, Gly97, Ser101, Gly102, Gly103, Ile107, Gly110, Met 124, Gly127, Gly128, Pro129, Leu135, Lys170, Tyr171, Pro172, Asp197, Met 199, Ser 204, Lys213, Tyr214, Gly215, and Ser221; or two or more of the amino acids listed above and Asp32, Ser33, Tyr104, Ala152, Asn155, Glu156, Gly166, Gly169, Phe189, Tyr217, and Met222 wherein both mutations cannot be made on the Asp32, Ser33, Tyr104, Ala152, Asn155, Glu156, Gly 166, Gly 169, Phe 189, Tyr217, and Met222 amino acids.

Another preferred BPN′ variant protease, hereafter referred to as “Protease D”, is described in WO 95/10615 published Apr. 20, 1995 by Genencor International as characterized by the wild-type BPN′ amino acid with mutation to position Asn76, in combination with mutations in one or more other amino acid positions selected from the group consisting of Asp99, Ser101, Gln103, Tyr104, Ser105, Ile107, Asn109, Asn123, Leu126, Gly127, Gly128, Leu135, Glu156, Gly166, Glu195, Asp197, Ser204, Gln206, Pro21O, Ala216, Tyr217, Asn218, Met222, Ser260, Lys265, and/or Ala274.

Another preferred BPN′ variant protease, hereafter referred to as “Protease F”, is described in U.S. Pat. No. 4,760,025, issued to Estell, et al. on Jul. 26, 1988 as characterized by the wild-type BPN′ amino acid with mutation to one or more amino acid positions selected from the group consisting of Asp32, Ser33, His64, Tyr104, Asn155, Glu156, Gly166, Gly169, Phe189, Tyr217, and Met222.

Preferred proteolytic enzymes, then, are selected from the group consisting of Alcalase®, BPN′, Protease A, Protease B, Protease D, and Protease F, and mixtures thereof. Protease F is most preferred.

Polyhydric Alcohol

The continuous phase of the oil in water emulsions suitable for use herein comprise at least one polyhydric alcohol in a concentration of less than 20%, preferably from about 0.5% to about 18%, more preferably from about 2% to about 15%, and especially from about 5% to about 12% by weight, of the polyhydric alcohol, or mixtures thereof.

For the purposes of this invention a polyhydric alcohol is considered any organic compound comprising two, or more, alcohol functions or alkoxylated derivatives thereof. In addition it is preferred that, if the composition has the form of an oil in water emulsion, that the polyhydric alcohol is present in the continuous phase.

Suitable polyhydric alcohols for use herein include polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, erythritol, threitol, pentaerythritol, xylitol, glucitol, mannitol, hexylene glycol, butylene glycol (e.g., 1,3-butylene glycol), hexane triol (e.g., 1,2,6-hexanetriol), trimethylol propane, neopentyl glycol, glycerine, ethoxylated glycerine, propane-1,3 diol, propoxylated glycerine and mixtures thereof. The alkoxylated derivatives of any of the above polyhydric alcohols are also suitable for use herein.

Preferred polyhydric alcohols of the present invention are selected from glycerine, butylene glycol, propylene glycol, dipropylene glycol, polyethylene glycol, hexane triol, ethoxylated glycerine and propoxylated glycerine, and mixtures thereof. Most preferred polyhydric alcohols for use in the present invention are glycerine, butylene glycol, propylene glycol, polyethylene glycol and mixtures thereof.

Optional Ingedients

The cosmetic compositions herein can comprise a wide variety of well known optional ingredients.

Carrier

The compositions of the present invention comprise a safe and effective amount of a dermatologically acceptable carrier, suitable for topical application to the skin or hair within which the essential materials and optional other materials are incorporated to enable the essential materials and optional components to be delivered to the skin or hair at an appropriate concentration. The carrier can thus act as a diluent, dispersant, solvent, or the like for the essential components which ensures that they can be applied to and distributed evenly over the selected target at an appropriate concentration.

The carrier, provided that it is suitable for inclusion within an oil in water emulsion, can be solid, semi-solid or liquid. Highly preferred carriers are liquid or semi-solid, such as creams, lotions and gels. Preferably the carrier is in the form of a lotion, cream or a gel, more preferably with sufficient thickness, or yield point, to prevent the particles from sedimenting. The carrier can itself be inert or it can possess dermatological benefits of its own. The carrier should also be physically and chemically compatible with the essential components described herein, and should not unduly impair stability, efficacy or other use benefits associated with the compositions of the present invention. The type of carrier utilised in the present invention depends on the type of product form desired for the composition. The topical compositions of the present invention may be made into a wide variety of product forms such as are known in the art. These include, but are not limited to, lotions, creams, gels, sticks, sprays, ointments, pastes, and mousses. These product forms may comprise several types of carriers including, but not limited to, solutions, emulsions, and gels.

Preferred carriers contain a dermatologically acceptable, hydrophilic diluent. Suitable hydrophilic diluents include water, organic hydrophilic diluents such as C₁-C₄ monohydric alcohols and low molecular weight glycols and polyols, including propylene glycol, polyethylene glycol (e.g. of MW 200-600), poly-propylene glycol (e.g. of MW 425-2025), glycerol, butylene glycol, butane-1,2,4-triol, hexane-1,2,6-triol, ethanol, iso-propanol, sorbitol esters, ethoxylated ethers, propoxylated ethers and combinations thereof. The diluent is preferably liquid. Water is an especially preferred diluent. The composition preferably comprises at least about 20% of the hydrophilic diluent.

Skin Care Active

A preferred ingredient the compositions herein comprise a skin care active at a level from about 0.1% to about 20%, preferably from about 1% to about 10%, more preferably from about 2% to about 8%, by weight.

The skin care active for use herein is selected from a vitamin B₃ component, panthenol, vitamin E, vitamin E acetate, retinol, retinyl propionate, retinyl palmitate, retinoic acid, vitamin C, theobromine, α-hydroxyacid, farnesol, phytantriol, salicylic acid, and mixtures thereof.

The preferred skin care active for use herein from the viewpoint of providing improved skin hydration is a vitamin B₃ component.

Vitamin B₃ Component

The compositions of the present invention preferably comprise from about 0.01% to about 20%, more preferably from about 0.1% to about 15%, even more preferably from about 0.5% to about 10%, and still more preferably from about 1% to about 8%, most preferably from about 1.5% to about 6%, of the vitamin B₃ compound.

As used herein, “vitamin B₃ compound” means a compound having the formula:

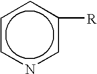

wherein R is —CONH₂ (i.e., niacinamide), —COOH (i.e., nicotinic acid) or —CH₂OH (i.e., nicotinyl alcohol); derivatives thereof; and salts of any of the foregoing. Exemplary derivatives of the foregoing vitamin B₃ compounds include nicotinic acid esters, including non-vasodilating esters of nicotinic acid, nicotinyl amino acids, nicotinyl alcohol esters of carboxylic acids, nicotinic acid N-oxide and niacinamide N-oxide.

Suitable esters of nicotinic acid include nicotinic acid esters of C₁-C₂₂, preferably C₁-C₁₆, more preferably C₁-C₆ alcohols. The alcohols are suitably straight-chain or branched chain, cyclic or acyclic, saturated or unsaturated (including aromatic), and substituted or unsubstituted. The esters are preferably non-vasodilating. As used herein, “non-vasodilating” means that the ester does not commonly yield a visible flushing response after application to the skin in the subject compositions (the majority of the general population would not experience a visible flushing response, although such compounds may cause vasodilation not visible to the naked eye). Non-vasodilating esters of nicotinic acid include tocopherol nicotinate and inositol hexanicotinate; tocopherol nicotinate is preferred. A more complete description of vitamin B₃ compounds is given in WO 98/22085.

Examples of the above vitamin B₃ compounds are well known in the art and are commercially available from a number of sources, e.g., the Sigma Chemical Company (St. Louis, Mo.); ICN Biomedicals, Inc. (Irvin, Calif.) and Aldrich Chemical Company (Milwaukee, Wis.). One or more vitamin B₃ compounds may be used herein. Preferred vitamin B₃ compounds are niacinamide and tocopherol nicotinate. Niacinamide is more preferred.

Retinoids

Another suitable skin care active is a retinoid. As used herein, “retinoid” includes all natural and/or synthetic analogs of Vitamin A or retinol-like compounds which possess the biological activity of Vitamin A in the skin as well as the geometric isomers and stereoisomers of these compounds.

The retinoid is preferably retinol, retinol esters (e.g., C₂-C₂₂ alkyl esters of retinol, including retinyl palmitate, retinyl acetate, retinyl proprionate), retinal, and/or retinoic acid (including all-trans retinoic acid and/or 13-cis-retinoic acid), more preferably retinoids other than retinoic acid. These compounds are well known in the art and are commercially available from a number of sources, e.g., Sigma Chemical Company (St. Louis, Mo.), and Boehringer Mannheim (Indianapolis, Ind.). Preferred retinoids are retinol, retinyl palmitate, retinyl acetate, retinyl proprionate, retinal, retinoic acid and combinations thereof. More preferred are retinol, retinoic propionate, retinoic acid and retinyl palmitate. The retinoid may be included as the substantially pure material, or as an extract obtained by suitable physical and/or chemical isolation from natural (e.g., plant) sources.

The compositions preferably contain from or about 0.005% to or about 2%, more preferably 0.01% to about 2% retinoid. Retinol is most preferably used in an amount of from or about 0.01% to or about 0.15%; retinol esters are most preferably used in an amount of from about 0.01% to about 2% (e.g., about 1%).

It is very highly preferred that the oil in water compositions suitable for use in the present invention comprise a vitamin complex consisting of from about 1% to about 5%, by weight, of vitamin B₃ compound or its derivatives; and from about 0.1% to about 1%, by weight, of a retinol compound or its derivatives in conjunction with from about 0.1% to about 1%, by weight, panthenol or its derivatives.

Additional Humectants

The compositions of the present invention may comprise additional humectants which are preferably present at a level of from about 0.01% to about 20%, more preferably from about 0.1% to about 15% and especially from about 0.5% to about 10%.

Preferred humectants include, but are not limited to, compounds selected from urea, D or DL panthenol, calcium pantothenate, royal jelly, panthetine, pantotheine, panthenyl ethyl ether, pangamic acid, pyridoxin, pantoyl lactose Vitamin B complex, hexane-1,2,6,-triol, guanidine or its derivatives. Highly preferred humectants are urea, panthenol and mixtures thereof. The above listed compounds may be incorporated singly or in combination.

Suitable additional humectants useful herein are sodium 2-pyrrolidone-5-carboxylate (NaPCA), guanidine; glycolic acid and glycolate salts (e.g. ammonium and quaternary alkyl ammonium); lactic acid and lactate salts (e.g. ammonium and quaternary alkyl ammonium); aloe vera in any of its variety of forms (e.g., aloe vera gel); hyaluronic acid and derivatives thereof (e.g., salt derivatives such as sodium hyaluronate); lactamide monoethanolamine; acetamide monoethanolamine; urea; panthenol and derivatives thereof; and mixtures thereof.

At least part (up to about 5% by weight of composition) of an additional humectant can be incorporated in the form of an admixture with a particulate cross-linked hydrophobic acrylate or methacrylate copolymer, itself preferably present in an amount of from about 0.1% to about 10%, which can be added either to the aqueous or disperse phase. This copolymer is particularly valuable for reducing shine and controlling oil while helping to provide effective moisturization benefits and is described in further detail by WO96/03964, incorporated herein by reference.

The above listed compounds may be incorporated singly or in combination. Preferred additional humectants are selected from urea, panthenol and mixtures thereof.

Emollients

The oil in water emulsions of the present invention generally comprise from about 1% to about 20%, preferably from about 1.5% to about 15%, more preferably from about 0.1% to about 8%, especially from about 0.5% to about 5% of a dermatologically acceptable emollient.

Emollients tend to lubricate the skin, increase the smoothness and suppleness of the skin, prevent or relieve dryness of the skin, and/or protect the skin. Emollients are typically water-immiscible, oily or waxy materials and emollients with high molecular weights can confer tacky properties to a topical composition. A wide variety of suitable emollients are known and may be used herein. Sagarin, Cosmetics, Science and Technology, 2nd Edition, Vol. 1, pp. 32-43 (1972), contains numerous examples of materials suitable as an emollient. All emollients discussed in application WO 00/24372 should be considered as suitable for use in the present invention although preferred examples are outlined in further detail below:

i) Straight and branched chain hydrocarbons having from about 7 to about 40 carbon atoms, such as dodecane, squalane, cholesterol, hydrogenated polyisobutylene, isohexadecane, isoeicosane, isooctahexacontane, isohexapentacontahectane, and the C₇-C₄₀ isoparaffins, which are C₇-C₄₀ branched hydrocarbons. Suitable branched chain hydrocarbons for use herein are selected from isopentacontaoctactane, petrolatum, and mixtures thereof. Suitable for use herein are branched chain aliphatic hydrocarbons sold under the trade name Permethyl (RTM) and commercially available from Presperse Inc., P.O. Box 735, South Plainfield, N.J. 07080, U.S.A.

ii) C₁-C₃₀ alcohol esters of C₁-C₃₀ carboxylic acids, C12-15 alkyl benzoates, and of C₂-C₃₀ dicarboxylic acids, e.g. isononyl isononanoate, isostearyl neopentanoate. isodecyl octanoate, isodecyl isononanoate, tridecyl isononanoate, myristyl octanoate, octyl pelargonate, octyl isononanoate, myristyl myristate, myristyl neopentanoate, myristyl octanoate, isopropyl myristate, myristyl propionate, isopropyl stearate, isopropyl isostearate, methyl isostearate, behenyl behenate, dioctyl maleate, diisopropyl adipate, and diisopropyl dilinoleate and mixtures thereof.

iii) C₁-C₃₀ mono- and poly-esters of sugars and related materials. These esters are derived from a sugar or polyol moiety and one or more carboxylic acid moieties. Depending on the constituent acid and sugar, these esters can be in either liquid or solid form at room temperature. Examples include: glucose tetraoleate, the galactose tetraesters of oleic acid, the sorbitol tetraoleate, sucrose tetraoleate, sucrose pentaoleate, sucrose hexaoleate, sucrose heptaoleate, sucrose octaoleate, sorbitol hexaester in which the carboxylic acid ester moieties are palmitoleate and arachidate in a 1:2 molar ratio, and the octaester of sucrose wherein the esterifying carboxylic acid moieties are laurate, linoleate and behenate in a 1:3:4 molar ratio. Other materials include cottonseed oil or soybean oil fatty acid esters of sucrose. Other examples of such materials are described in WO 96/16636, incorporated by reference herein. A particularly preferred material is known by the INCI name sucrose polycottonseedate

iv) Vegetable oils and hydrogenated vegetable oils. Examples of vegetable oils and hydrogenated vegetable oils include safflower oil, coconut oil, cottonseed oil, menhaden oil, palm kernel oil, palm oil, peanut oil, soybean oil, rapeseed oil, linseed oil, rice bran oil, pine oil, sesame oil, sunflower seed oil, partially and fully hydrogenated oils from the foregoing sources, and mixtures thereof

v) Soluble or colloidally-soluble moisturising agents. Examples include hylaronic acid and starch-grafted sodium polyacrylates such as Sanwet (RTM) IM-1000, IM-1500 and IM-2500 available from Celanese Superabsorbent Materials, Portsmith, Va., USA and described in U.S. Pat. No. 4,076,663.

Preferred emollients for use herein are isohexadecane, isooctacontane, petrolatum, isononyl isononanoate, isodecyl octanoate, isodecyl isononanoate, tridecyl isononanoate, myristyl octanoate, octyl isononanoate, myristyl myristate, methyl isostearate, isopropyl isostearate, C12-15 alkyl benzoates and mixtures thereof. Particularly preferred emollients for use herein are isohexadecane, isononyl isononanoate, methyl isostearate, isopropyl isostearate, petrolatum, or mixtures thereof. Due to its poor skin feel properties castor oil is not a preferred emollient for use herein.

Emulsifiers/Surfactants

Compositions herein preferably contain an emulsifier and/or surfactant, generally to help disperse and suspend the disperse phase within the continuous aqueous phase. A surfactant may also be useful if the product is intended for skin cleansing. For convenience hereinafter emulsifiers will be referred to under the term ‘surfactants’, thus ‘surfactant(s)’ will be used to refer to surface active agents whether used as emulsifiers or for other surfactant purposes such as skin cleansing. Known or conventional surfactants can be used in the composition, provided that the selected agent is chemically and physically compatible with essential components of the composition, and provides the desired characteristics. Suitable surfactants include non-silicone derived materials, and mixtures thereof. All surfactants discussed in application WO 00/24372 should be considered as suitable for use in the present invention.

The compositions of the present invention preferably comprise from about 0.05% to about 15% of a surfactant or mixture of surfactants. The exact surfactant or surfactant mixture chosen will depend upon the pH of the composition and the other components present.

Preferred surfactants are nonionic. Among the nonionic surfactants that are useful herein are those that can be broadly defined as condensation products of long chain alcohols, e.g. C₈₋₃₀ alcohols, with sugar or starch polymers ie glycosides. Other useful nonionic surfactants include the condensation products of alkylene oxides with fatty acids (i.e. alkylene oxide esters of fatty acids). These materials have the general formula RCO(X)ₙOH wherein R is a C₁₀₋₃₀ alkyl group, X is —OCH₂CH₂— (i.e. derived from ethylene glycol or oxide) or —OCH₂CHCH₃— (i.e. derived from propylene glycol or oxide), and n is an integer from about 6 to about 200. Other nonionic surfactants are the condensation products of alkylene oxides with 2 moles of fatty acids (i.e. alkylene oxide diesters of fatty acids). These materials have the general formula RCO(X)ₙOOCR wherein R is a C₁₀₋₃₀ alkyl group, X is —OCH₂CH₂— (i.e. derived from ethylene glycol or oxide) or —OCH₂CHCH₃— (i.e. derived from propylene glycol or oxide), and n is an integer from about 6 to about 100. An emulsifier for use herein is most preferably a fatty acid ester blend based on a mixture of sorbitan fatty acid ester and sucrose fatty acid ester, especially a blend of sorbiton stearate and sucrose cocoate. This is commercially available from ICI under the trade name Arlatone 2121. Even further suitable examples include a mixture of cetearyl alcohols, cetearyl glucosides such as those available under the trade name Montanov 68 from Seppic and Emulgade PL68/50 available from Henkel.

The hydrophilic surfactants useful herein can alternatively or additionally include any of a wide variety of cationic, anionic, zwitterionic, and amphoteric surfactants such as are known in the art. See, e.g., McCutcheon's, Detergents and Emulsifiers, North American Edition (1986), published by Allured Publishing Corporation; U.S. Pat. No. 5,011,681 to Ciotti et al., issued Apr. 30, 1991; U.S. Pat. No. 4,421,769 to Dixon et al., issued Dec. 20, 1983; and U.S. Pat. No. 3,755,560 to Dickert et al., issued Aug. 28, 1973. A wide variety of anionic surfactants are also useful herein. See, e.g., U.S. Pat. No. 3,929,678, to Laughlin et al., issued Dec. 30, 1975.

A wide variety of anionic surfactants are also useful herein. See, e.g., U.S. Pat. No. 3,929,678, to Laughlin et al., issued Dec. 30, 1975. Exemplary anionic surfactants include the alkoyl isethionates (e.g., C₁₂-C₃₀), alkyl and alkyl ether sulfates and salts thereof, alkyl and alkyl ether phosphates and salts thereof, alkyl methyl taurates (e.g., C₁₂-C₃₀), and soaps (e.g., alkali metal salts, e.g., sodium or potassium salts) of fatty acids.

Amphoteric and zwitterionic surfactants are also useful herein. Examples of amphoteric and zwitterionic surfactants which can be used in the compositions of the present invention are those which are broadly described as derivatives of aliphatic secondary and tertiary amines in which the aliphatic radical can be straight or branched chain and wherein one of the aliphatic substituents contains from about 8 to about 22 carbon atoms (preferably C₈-C₁₈) and one contains an anionic water solubilising group, e.g., carboxy, sulfonate, sulfate, phosphate, or phosphonate. Examples are alkyl imino acetates, and iminodialkanoates and aminoalkanoates, imidazolinium and ammonium derivatives. Other suitable amphoteric and zwitterionic surfactants are those selected from the group consisting of betaines, sultaines, hydroxysultaines, and branched and unbranched alkanoyl sarcosinates, and mixtures thereof.

Preferred emulsions of the present invention include a silicone containing emulsifier or surfactant. A wide variety of silicone emulsifiers are useful herein. These silicone emulsifiers are typically organically modified organopolysiloxanes, also known to those skilled in the art as silicone surfactants. Useful silicone emulsifiers include dimethicone copolyols. These materials are polydimethyl siloxanes which have been modified to include polyether side chains such as polyethylene oxide chains, polypropylene oxide chains, mixtures of these chains, and polyether chains containing moieties derived from both ethylene oxide and propylene oxide. Other examples include alkyl-modified dimethicone-copolyols, i.e., compounds which contain C₂-C₃₀ pendant side chains. Still other useful dimethicone copolyols include materials having various cationic, anionic, amphoteric, and zwitterionic pendant moieties.

Polymeric Thickening Agents

The compositions of the present invention can comprise at least one polymeric thickening agent.

The polymeric thickening agents useful herein preferably have a number average molecular weight of greater than 20,000, more preferably greater than 50,000 and especially greater than 100,000.

In general, the compositions of the present invention may comprise from about 0.01% to about 10%, preferably from about 0.1% to about 8% and most preferably from about 0.5% to about 5% by weight of the composition of the polymeric thickening agent, or mixtures thereof.

Preferred polymer thickening agents for use herein include non-ionic thickening agents and anionic thickening agents, or mixtures thereof. Suitable non-ionic thickening agents include polyacrylamide polymers, crosslinked poly(N-vinylpyrrolidones), polysaccharides, natural or synthetic gums, polyvinylpyrrolidone, and polyvinylalcohol. Suitable anionic thickening agents include acrylic acid/ethyl acrylate copolymers, carboxyvinyl polymers and crosslinked copolymers of alkyl vinyl ethers and maleic anhydride. Particularly preferred thickening agents for use herein are the non-ionic polyacrylamide polymers such as polyacrylamide and isoparaffin and laureth-7, available under the trade name Sepigel 305 from Seppic Corporation, and acrylic acid/ethyl acrylate copolymers and the carboxyvinyl polymers sold by the B. F. Goodrich Company under the trade mark of Carbopol resins, or mixtures thereof. Suitable Carbopol resins may be hydrophobically modified, and other suitable resins are described in WO98/22085, or mixtures thereof.

Silicone Oil

The present compositions preferably comprise, at least one silicone oil phase. Silicone oil phase(s) generally comprises from about 0.1% to about 20%, preferably from about 0.5% to about 10%, more preferably from about 0.5% to about 5%, of the composition. The, or each, silicone oil phase preferably comprises one or more silicone components.

Silicone components can be fluids, including straight chain, branched and cyclic silicones. Suitable silicone fluids useful herein include silicones inclusive of polyalkyl siloxane fluids, polyaryl siloxane fluids, cyclic and linear polyalkylsiloxanes, polyalkoxylated silicones, amino and quaternary ammonium modified silicones, polyalkylaryl siloxanes or a polyether siloxane copolymer and mixtures thereof. The silicone fluids can be volatile or non-volatile. Silicone fluids generally have a weight average molecular weight of less than about 200,000. Suitable silicone fluids have a molecular weight of about 100,000 or less, preferably about 50,000 or less, most preferably about 10,000 or less. Preferably the silicone fluid is selected from silicone fluids having a weight average molecular weight in the range from about 100 to about 50,000 and preferably from about 200 to about 40,000. Typically, silicone fluids have a viscosity ranging from about 0.65 to about 600,000 mm².s⁻¹, preferably from about 0.65 to about 10,000 mm².s⁻¹ at 25° C. The viscosity can be measured by means of a glass capillary viscometer as set forth in Dow Corning Corporate Test Method CTM0004, Jul. 29, 1970. Suitable polydimethyl siloxanes that can be used herein include those available, for example, from the General Electric Company as the SF and Viscasil (RTM) series and from Dow Corning as the Dow Corning 200 series. Also useful are essentially non-volatile polyalkylarylsiloxanes, for example, polymethylphenylsiloxanes, having viscosities of about 0.65 to 30,000 mm².s⁻¹ at 25° C. These siloxanes are available, for example, from the General Electric Company as SF 1075 methyl phenyl fluid or from Dow Corning as 556 Cosmetic Grade Fluid. Cyclic polydimethylsiloxanes suitable for use herein are those having a ring structure incorporating from about 3 to about 7 (CH₃)₂SiO moieties.

In preferred embodiments, the silicone fluid is selected from dimethicone, decamethylcyclopentasiloxane, octamethylcyclotetrasiloxane, phenyl methicone, and mixtures thereof.

Silicone gums can also be used herein. The term “silicone gum” herein means high molecular weight silicones having a weight average molecular weight in excess of about 200,000 and preferably from about 200,000 to about 4,000,000. Iincluded are non-volatile polyalkyl and polyaryl siloxane gums. In preferred embodiments, a silicone oil phase comprises a silicone gum or a mixture of silicones including the silicone gum. Typically, silicone gums have a viscosity at 25° C. in excess of about 1,000,000 mm²s⁻¹. The silicone gums include dimethicones as described by Petrarch and others including U.S. Pat. No. 4,152,416, May 1, 1979 to Spitzer, et al, and Noll, Walter, Chemistry and Technology of Silicones, New York: Academic Press 1968. Also describing silicone gums are General Electric Silicone Rubber Product Data Sheets SE 30, SE 33, SE 54 and SE 76. Specific examples of silicone gums include polydimethylsiloxane, (polydimethylsiloxane)(methylvinylsiloxane) copolymer, poly(dimethylsiloxane-(diphenyl)(methylvinylsiloxane) copolymer and mixtures thereof. Preferred silicone gums for use herein are silicone gums having a molecular weight of from about 200,000 to about 4,000,000 selected from dimethiconol, and dimethicone and mixtures thereof.

A silicone phase herein preferably comprises a silicone gum incorporated into the composition as part of a silicone gum-fluid blend. When the silicone gum is incorporated as part of a silicone gum-fluid blend, the silicone gum preferably constitutes from about 5% to about 40%, especially from about 10% to 20% by weight of the silicone gum-fluid blend. Suitable silicone gum-fluid blends herein are mixtures consisting essentially of:

(i) a silicone having a molecular weight of from about 200,000 to about 4,000,000 selected from dimethiconol, fluorosilicone and dimethicone and mixtures thereof; and

(ii) a carrier which is a silicone fluid, the carrier having a viscosity from about 0.65 mm².s⁻¹ to about 100 mm².s⁻¹,

wherein the ratio of i) to ii) is from about 10:90 to about 20:80 and wherein said silicone gum-based component has a final viscosity of from about 100 mm².s⁻¹ to about 100,000 mm².s⁻¹, preferably from 500 mm².s⁻¹ to about 10,000 mm².s⁻¹.

An especially preferred silicone-gum fluid blend based component for use in the compositions herein is a dimethiconol gum having a molecular weight of from about 200,000 to about 4,000,000 along with a silicone fluid carrier with a viscosity of about 0.65 to 100 mm².s⁻¹. An example of this silicone component is Dow Corning Q2-1403 (85% 5 mm².s⁻¹ Dimethicone Fluid/15% Dimethiconol) and Dow Corning Q2-1401 available from Dow Corning.

Further silicone components suitable for use in a silicone oil phase herein are crosslinked polyorganosiloxane polymers, optionally dispersed in a fluid carrier. In general, when present the crosslinked polyorganosiloxane polymers, together with its carrier (if present) comprises 0.1% to about 20%, preferably from about 0.5% to about 10%, more preferably from about 0.5% to about 5% of the composition. Such polymers comprise polyorganosiloxane polymers crosslinked by a crosslinking agent. Suitable crosslinking agents are disclosed in WO98/22085. Examples of suitable polyorganosiloxane polymers for use herein include methyl vinyl dimethicone, methyl vinyl diphenyl dimethicone and methyl vinyl phenyl methyl diphenyl dimethicone.

Specific commercially available crosslinked polyorganosiloxane polymers for use herein are silicone vinyl crosspolymer mixtures available under the tradename KSG supplied by Shinetsu Chemical Co., Ltd, for example KSG-15, KSG-16, KSG-17, KSG-18. These materials contain a combination of crosslinked polyorganosiloxane polymer and silicone fluid. Particularly preferred for use herein especially in combination with the organic amphiphilic emulsifier material is KSG-18. The assigned INCI names for KSG-15, KSG-16, KSG-17 and KSG-18 are cyclomethicone dimethicone/vinyl dimethicone crosspolymer, dimethicone dimethicone/vinyl dimethicone crosspolymer, cyclomethicone dimethicone/vinyl dimethicone crosspolymer and phenyl trimethicone dimethicone/phenyl vinyl dimethicone crosspolymer, respectively.

Another class of silicone components suitable for use in a silicone oil phase herein includes polydiorganosiloxane-polyoxyalkylene copolymers containing at least one polydiorganosiloxane segment and at least one polyoxyalkylene segment. Suitable polydiorganosiloxane segments and copolymers thereof are disclosed in WO98/22085. Suitable polydiorganosiloxane-polyalkylene copolymers are available commercially under the tradenames Belsil (RTM) from Wacker-Chemie GmbH, Geschaftsbereich S, Postfach D-8000 Munich 22 and Abil (RTM) from Th. Goldschmidt Ltd., Tego House, Victoria Road, Ruislip, Middlesex, HA4 OYL, for example Belsil (RTM) 6031 and Abil (RTM) B88183. A particularly preferred copolymer fluid blend for use herein includes Dow Corning DC3225C which has the CTFA designation Dimethicone/Dimethicone copolyol.

Sunscreens

Compositions of the present invention preferably comprise an organic sunscreen. Suitable sunscreens can have UVA absorbing properties, UVB absorbing properties or a mixture thereof. The exact amount of the sunscreen active will vary depending upon the desired Sun Protection Factor, ie the “SPF” of the composition as well as the desired level of UV protection. The compositions of the present invention preferably comprise an SPF of at least 10, preferably at least 15. SPF is a commonly used measure of photoprotection of a sunscreen against erythema. The SPF is defined as a ratio of the ultraviolet energy required to produce minimal erythema on protected skin to that required to products the same minimal erythema on unprotected skin in the same individual. See Federal Register, 43, No 166, pp. 38206-38269, Aug. 25, 1978). Amounts of the sunscreen used are typically from about 2% to about 20%, more typically from about 4% to about 14%. Suitable sunscreens include, but are not limited to, those found in the CTFA International Cosmetic Ingredient Dictionary and Handbook, 7^{th} edition, volume 2 pp. 1672, edited by Wenninger and McEwen (The Cosmetic, Toiletry, and Fragrance Association, Inc., Washington, D.C., 1997).

The compositions of the present invention preferably comprise a UVA absorbing sunscreen actives which absorb UV radiation having a wavelength of from about 320 nm to about 400 nm. Suitable UVA absorbing sunscreen actives are selected from dibenzoylmethane derivatives, anthranilate derivatives such as methylanthranilate and homomethyl, 1-N-acetylanthranilate, and mixtures thereof. Examples of dibenzoylmethane sunscreen actives are described in U.S. Pat. No. 4,387,089 issued to Depolo; and in Sunscreens: Development, Evaluation, and Regulatory Aspects edited by N. J. Lowe and N. A. Shaath, Marcel Dekker, Inc (1990). The UVA absorbing sunscreen active is preferably present in an amount to provide broad spectrum UVA protection either independently, or in combination with, other UV protective actives which may be present in the composition.

Preferred UVA sunscreen actives are dibenzoylmethane sunscreen actives and their derivatives. They include, but are not limited to, those selected from 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropylbenzoylmethane, 4-(1,1-dimethylethyl)-4′-methoxydibenzoylmethane, 2-methyl-5-isopropyl-4′-methoxydibenzoylmethane, 2-methyl-5-tert-butyl-4′-methoxy-dibenzoylmethane, 2,4-dimethyl-4′-methoxydibenzoylmethane, 2,6-dimethyl-4′-tert-butyl-4′methoxydibenzoylmethane, and mixtures thereof. Preferred dibenzoyl sunscreen actives include those selected from 4-(1,1-dimethylethyl)-4′-methoxydibenzoylmethane, 4-isopropyldibenzoylmethane, and mixtures thereof. A more preferred sunscreen active is 4-(1,1-dimethylethyl)-4′-methoxydibenzoylmethane.

The sunscreen active 4-(1,1-dimethylethyl)-4′-methoxydibenzoylmethane, which is also known as butyl methoxydibenzoylmethane or Avobenzone, is commercially available under the names of Parsol® 1789 from Givaudan Roure (International) S. A. (Basel, Switzerland) and Eusolex® 9020 from Merck & Co., Inc (Whitehouse Station, N.J.). The sunscreen 4-isoproplydibenzoylmethane, which is also known as isopropyldibenzoylmethane, is commercially available from Merck under the name of Eusolex® 8020.

The compositions of the present invention preferably further comprise a UVB sunscreen active which absorbs UV radiation having a wavelength of from about 290 nm to abut 320 nm. The compositions comprise an amount of the UVB sunscreen active which is safe and effective to provide UVB protection either independently, or in combination with, other UV protective actives which may be present in the compositions. The compositions preferably comprise from about 0.1% to abut 16%, more preferably from about 0.1% to about 12%, and most preferably from about 0.5% to about 8% by weight, of UVB absorbing organic sunscreen.

A wide variety of UVB sunscreen actives are suitable for use herein. Nonlimiting examples of such organic sunscreen actives are described in U.S. Pat. No. 5,087,372 issued Feb. 11, 1992 to Haffey et al.; and U.S. Pat. Nos. 5,073,371 and 5,073,372 both issued on Dec. 17, 1991 to Turner et al. and Segarin, et al., at Chapter VIII, pages 189 et seq., of Cosmetics Science and Technology. Still other useful sunscreens are those disclosed in U.S. Pat. No. 4,937,370, to Sabatelli, issued Jun. 26, 1990; and U.S. Pat. No. 4,999,186, to Sabatelli et al., issued Mar. 12, 1991. Preferred UVB sunscreen actives are selected from 2-ethylhexyl-2-cyano-3,2-ethylhexyl N,N-dimethyl-p-aminobenzoate, p-aminobenzoic acid, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, 3-diphenylacrylate (referred to as octocrylene), 2-phenyl-benzimidazole-5-sulphonic acid (PBSA), cinnamates and their derivatives such as 2-ethylhexyl-p-methoxycinnamate and octyl-p-methoxycinnamate, TEA salicylate, octyldimethyl PABA, camphor derivatives and their derivatives, and mixtures thereof. Preferred organic sunscreen actives are 2-ethylhexyl-2-cyano-3,3-diphenylacrylate (referred to as octocrylene), 2-phenyl-benzimidazole-5-sulphonic acid (PBSA), octyl-p-methoxycinnamate, and mixtures thereof. Salt and acid neutralised forms of the acidic sunscreens are also useful herein.

An agent may also be added to any of the compositions useful in the present invention to stabilise the UVA sunscreen to prevent it from photo-degrading on exposure to UV radiation and thereby maintaining its UVA protection efficacy. A wide range of compounds have been cited as providing these stabilising properties and should be chosen to compliment both the UVA sunscreen and the composition as a whole. Suitable stabilising agents include, but are not limited to, those described in U.S. Pat. Nos. 5,972,316; 5,968,485; 5,935,556; 5,827,508 and Patent WO 00/06110. Preferred examples of stabilising agents for use in the present invention include 2-ethylhexyl-2-cyano-3,3-diphenylacrylate (referred to as octocrylene), ethyl-2-cyano-3,3-diphenylacrylate, 2-ethylhexyl-3,3-diphenylacrylate, ethyl-3,3-bis(4-methoxyphenyl)acrylate, and mixtures thereof. 2-ethylhexyl-2-cyano-3,3-diphenylacrylate is most preferred.

An agent may also be added to any of the compositions useful in the present invention to improve the skin substantivity of those compositions, particularly to enhance their resistance to being washed off by water, or rubbed off. A preferred agent which will provide this benefit is a copolymer of ethylene and acrylic acid. Compositions comprising this copolymer are disclosed in U.S. Pat. No. 4,663,157, Brock, issued May 5, 1987.

In addition to the organic sunscreens compositions of the present invention can additionally comprise inorganic physical sunblocks. Nonlimiting examples of suitable physical sunblocks are described in CTFA International Cosmetic Ingredient Dictionary, 6^{th} Edition, 1995, pp. 1026-28 and 1103, Sayre, R. M. et al., “Physical Sunscreens”, J. Soc. Cosmet. Chem., vol 41, no 2, pp. 103-109 (1990). Preferred inorganic physical sunblocks are zinc oxide and titanium dioxide, and mixtures thereof.

When used, the physical sunblocks are present in an amount such that the present compositions are transparent on the skin (ie non-whitening), preferably less than or equal to about 5%. When titanium dioxide is used, it can have an anatase, rutile, or amorphous structure. Physical sunblock particles, eg titanium dioxide and zinc oxide, can be uncoated or coated with a variety of materials including but not limited to amino acids, aluminium compounds such as alumina, aluminium stearate, aluminium laurate, and the like; carboxylic acids and their salts eg stearic acid and its salts; phospholipids such as lecithin; organic silicone compounds; inorganic silicone compounds such as silica and silicates; and mixtures thereof. A preferred titanium dioxide is commercially available from Tayca (Japan) and is distributed by Tri-K Industries (Emerson, N.J.) under the MT micro-ionised series (eg MT 1 OOSAS).

The compositions of the present invention preferably comprise from about 0.1% to about 10%, more preferably from about 0.1% to about 4%, and most preferably from about 0.5% to about 2.5%, by weight, of inorganic sunscreen.

A wide variety of optional ingredients such as neutralising agents, perfumes, and colouring agents, can also be added to the compositions herein. It is preferred that any additional ingredients enhance the skin softness/smoothness benefits of the product. In addition it is preferred that any such ingredients do not negatively impact the aesthetic properties of the product. As such high levels of proteins such as collagen and elastin are not preferred in compositions useful in the present invention.

The compositions of the invention can also contain from about 0.01% to about 10%, preferably from about 0.1% to about 5% of a panthenol moisturizer. The panthenol moisturizer can be selected from D-panthenol ([R]-2,4-dihydroxy-N-[3-hydroxypropyl)]-3,3-dimethylbutamide), DL-panthenol, calcium pantothenate, royal jelly, panthetine, pantotheine, panthenyl ethyl ether, pangamic acid, pyridoxin, and pantoyl lactose.

In a preferred embodiment, the compositions of the present invention additionally comprise a salt selected from alkali metal and alkaline earth metal salts, and mixtures thereof, preferably sodium, calcium and magnesium salts, and mixtures thereof. Especially preferred for use herein are calcium and magnesium salts. The compositions herein preferably comprise from about 5 ppm to about 500 ppm of the salt, based on the amount of metal ion.

In a further preferred embodiment, the compositions herein may comprise additional enzymes selected from lipases, phospholipases, glycosidases, lactoperoxidases and cellulases, and mixtures thereof.

Neutralizing agents suitable for use in neutralizing acidic group containing hydrophilic gelling agents herein include sodium hydroxide, potassium hydroxide, ammonium hydroxide, monoethanolamine, diethanolamine, amino methyl propanol, tris-buffer and triethanolamine.

Other optional materials include keratolytic agents; water-soluble or solubilizable preservatives preferably at a level of from about 0.1% to about 5%, such as Germall 115, methyl, ethyl, propyl and butyl esters of hydroxybenzoic acid, benzyl alcohol, DMDM hydantoin iodopropanyl butylcarbanate available under the trade name Glydant Plus from Lonza, EDTA, Euxyl (RTM) K400, Bromopol (2-bromo-2-nitropropane-1,3-diol) and phenoxypropanol; anti-bacterials such as Irgasan (RTM) and phenoxyethanol (preferably at levels of from 0.1% to about 5%); soluble or colloidally-soluble moisturising agents such as hylaronic acid and starch-grafted sodium polyacrylates such as Sanwet (RTM) IM-1000, IM-1500 and IM-2500 available from Celanese Superabsorbent Materials, Portsmith, Va., USA and described in USA-A-4,076,663; vitamins such as vitamin A, vitamin C, vitamin E and derivatives thereof and building blocks thereof such as phytantriol and vitamin K and components thereof such as the fatty alcohol dodecatrienol; alpha and beta hydroxyacids; aloe vera; sphingosines and phytosphingosines, cholesterol; skin whitening agents; N-acetyl cysteine; colouring agents; antibacterial agents such as TCC/TCS, also known as triclosan and trichlorocarbon; perfumes and perfume solubilizers. Examples of alpha hydroxy acids include glycolic acid, lactic acid, malic acid, citric acid, glycolic acid in conjunction with ammonium glycolate, alpha-hydroxy ethanoic acid, alpha-hydroxyoctanoic acid, alpha-hydroxycaprylic acid, hydroxycaprylic acid, mixed fruit acid, tri-alp0ha hydroxy fruit acids, triple fruit acid, sugar cane extract, alpha hydroxy and botanical comprise, 1-alpha hydroxy acid and glycomer in crosslinked fatty acids alpha nutrium. Preferred examples of alpha hydroxy acids are glycolic acid and lactic acid. It is preferred that alpha hydroxy acids are used in levels of upto 10%.

The compositions of the present invention can additionally comprise from about 0.1% to about 5% by weight of aluminium starch octenylsuccinate. Aluminium starch octenylsuccinate is the aluminium salt of the reaction product of octenylsuccinic anhydride with starch and is commercially available under the trade name from Dry Flo National Starch & Chemical Ltd. Dry Flo is useful herein from the viewpoint of skin feel and application characteristics.

A safe and effective amount of an anti-inflammatory agent may be added to the compositions of the subject invention, preferably from about 0.1% to about 5%, more preferably from about 0.1% to about 2%, of the composition. The anti-inflammatory agent enhances the skin appearance benefits of the present invention, e.g., such agents contribute to a more uniform and acceptable skin tone or colour. The exact amount of anti-inflammatory agent to be used in the compositions will depend on the particular anti-inflammatory agent utilised since such agents vary widely in potency.

Compositions of the subject invention can further include an anti-oxidant/radical scavenger. The anti-oxidant/radical scavenger is especially useful for providing protection against UV radiation which can cause increased scaling or texture changes in the stratum corneum and against other environmental agents which can cause skin damage. Suitable amounts are from about 0.1% to about 10%, more preferably from about 1% to about 5%, of the composition. Anti-oxidants/radical scavengers such as ascorbic acid (vitamin C) and its salts.

The inclusion of a chelating agent is especially useful for providing protection against UV radiation which can contribute to excessive scaling or skin texture changes and against other environmental agents which can cause skin damage. A suitable amount is from about 0.01% to about 1%, more preferably from about 0.05% to about 0.5%, of the composition. Exemplary chelators that are useful herein are disclosed in U.S. Pat. No. 5,487,884, incorporated herein by reference. Preferred chelators useful in compositions of the subject invention are ethylenediamine tetraacetic acid (EDTA), furildioxime, and derivatives thereof.

The compositions of the present invention can also comprise a skin lightening agent. When used, the compositions preferably comprise from about 0.1% to about 10%, more preferably from about 0.2% to about 5%, also preferably from about 0.5% to about 2%, of a skin lightening agent. Suitable skin lightening agents include those known in the art, including kojic acid, arbutin, ascorbic acid and derivatives thereof, e.g., magnesium ascorbyl phosphate. Further skin lightening agents suitable for use herein also include those described in WO 95/34280 and WO 95/23780; each incorporated herein by reference.

Other optional materials include water-soluble or solubilizable preservatives preferably at a level of from about 0.1% to about 5%, such as Germall 115, methyl, ethyl, propyl and butyl esters of hydroxybenzoic acid, benzyl alcohol, DMDM hydantoin iodopropanyl butylcarbanate available under the trade name Glydant Plus from Lonza, EDTA, Euxyl (RTM) K400, Bromopol (2-bromo-2-nitropropane-1,3-diol) and phenoxypropanol; anti-bacterials such as Irgasan (RTM) and phenoxyethanol (preferably at levels of from 0.1% to about 5%). Antibacterial agents such as TCC/TCS, also known as triclosan and trichlorocarbon are also useful in compositions of the present invention.

Other optional materials herein include pigments which, where water-insoluble, contribute to and are included in the total level of oil phase ingredients. Pigments suitable for use in the compositions of the present invention can be organic and/or inorganic. Also included within the term pigment are materials having a low colour or lustre such as matte finishing agents, and also light scattering agents. Preferably the compositions of the present invention comprise particulate materials having a refractive index of from about 1.3 to about 1.7, the particulate materials being dispersed in the composition and having a median particle size of from about 2 to about 30 μm. Preferably the particulates useful herein have relatively narrow distributions, by which is meant that more than 50% of the particles fall within 3 μm either side of the respective median value. Also preferred is that more than 50%, preferably more than 60%, more preferably more than 70% of particles fall within the size ranges prescribed for the respective median values. Suitable particulate materials are organic or organosilicone and preferably organosilicone polymers. Preferred particles are free-flowing, solid, materials. By “solid” is meant that the particles are not hollow. The void at the centre of hollow particles can have an adverse effect on refractive index and therefore the visual effects of the particles on either skin or the composition. Suitable organic particulate materials include those made of polymethylsilsesquioxane, referenced above, polyamide, polythene, polyacrylonitrile, polyacrylic acid, polymethacrylic acid, polystyrene, polytetrafluoroethylene (PTFE) and poly(vinylidene chloride). Copolymers derived from monomers of the aforementioned materials can also be used. Inorganic materials include silica and boron nitride. Representative commercially available examples of useful particulate materials herein are Tospearl® 145 which has a median particle size of about 4.5 μm and EA-209® from Kobo which is an ethylene/acrylic acid copolymer having a median particle size of about 10 μm, Nylon-12 available under the trade name Orgasol 2002 from Elf Atochem, France, or mixtures thereof.

Further examples of suitable pigments are titanium dioxide, predispersed titanium dioxide from Kobo e.g. Kobo GWL75CAP, iron oxides, acyglutamate iron oxides, ultramarine blue, D&C dyes, carmine, and mixtures thereof. Depending upon the type of composition, a mixture of pigments will normally be used. The preferred pigments for use herein from the viewpoint of moisturisation, skin feel, skin appearance and emulsion compatibility are treated pigments. The pigments can be treated with compounds such as amino acids, silicones, lecithin and ester oils.

The compositions of the invention are generally in emulsion form and are preferably formulated so as to have a product viscosity of at least about 4,000 mPa.s and preferably in the range from about 4,000 to about 1,000,000 mPa.s, more preferably from about 8,000 to about 350,000 mPa.s and especially from about 10,000 to about 250,000 mPa.s and even more especially from about 10,000 to about 150,000 mPa.s (25° C., neat, Brookfield RVT, T Spindle at 5 rpms and Heliopath Stand).

Packaging

It is highly preferred that oil in water compositions of the present invention is packaged in a pack comprising two or more chambers wherein the first chamber comprises an aqueous composition comprising:

(a) from about from about 0.0001% to about 10%, preferably from about 0.01% to about 5%, more preferably from about 0.05% to about 2%, by weight, protease enzyme;

(b) from about 20% to about 99%, preferably from about 50% to about 98%, more preferably from about 60 to about 95%, by weight, of a polyhydric alcohol; and

(c) less than about 20% and preferably less than about 12%, by weight, of water;

and wherein the second chamber comprises a placebo composition as an oil in water emulsion and wherein the compositions from the two chambers are dispensed from the dispensing system simultaneously, preferably through a single nozzle. As such it is possible to store the protease enzyme in highly stable conditions comprising high level of polyhydric alcohol whilst simultaneously dispensing a final composition in the form of an oil in water emulsion.

By altering the dispensing ratio of the pack it is possible to control the level of enzyme that is dispensed with each dose. Furthermore when the compositions of the two chambers are dispensed they mix which results in a dilution of the enzyme storage composition and thus an increase in water activity which reactivates the enzyme. In addition, this dilution of the stored enzyme composition, means that the polyhydric alcohol levels of the final composition are low enough that the product has acceptable aesthetics. It is preferred that the weight ratio of the composition dispensed from the first chamber of the multi chambered dispensing system and the second chamber of the multi chambered dispensing system is in the range from about 1:1 to about 1:50, preferably from about 1:5 to about 1:30, more preferably from about 1:10 to about 1:25 and most preferably from about 1:15 to about 1:23.

Further examples of the pack are disclosed in patent WO 97/27841 which is incorporated herein by reference. Highly preferred is pack the Symbio pack, available from Airspray International.

It is preferred that when the compositions of the present invention are stored in such a multi chambered pack that the composition of the first chamber comprising the enzyme also comprises from about 50 to about 400 ppm of a metal salt selected from the group consisting of alkali metal salts, alkaline earth metal salts and mixtures thereof. It is preferred that the metal salts are selected from salts of sodium, calcium, magnesium and mixtures thereof.

Method of Use

Compositions of the present invention are useful for providing superior desquamation/skin exfoliation and thus improved skin softness benefits. However, as mentioned previously, there is a risk that, if consumers are overexposed to the protease enzymes or misuse the product, that sensitisation can occur. As such it is responsible to minimise the level of enzyme exposure. This can be achieved by formulating the final composition to comprise low levels of enzyme but also by limiting exposure by introducing a usage regimen.

It is highly preferred a cosmetic method for treating the skin and/or hair which comprises administering a daily sequence of one or more unit doses of topical cosmetic composition over a repeating cycles is recommended for use of compositions of the present invention. The preferred dosage sequence of the cycle comprises:

(a) administering, as phase one, for a period of 1 to 365, preferably from 1 to 50, more preferably from 1 to 10, even more preferably from 1 to 7 and most preferably from 3 to 7 days a cosmetic composition comprising a protease enzyme; followed by

(b) administering, as phase two, for a period of 1 to 365, preferably from 1 to 100, more preferably from 1 to 50, even more preferably from 10 to 40 and most preferably from 20 to 30 days a placebo, or maintenance, composition.

The cosmetic method of treatment is further characterised in that the dosage cycle is repeated for from about 1 to 1000 times, preferably from about 1 to 100 times, more preferably from about 1 to 12 times, even more preferably from about 1 to 6 times and most preferably from about 1 to 3 times in a single treatment period.

Furthermore each daily dosage sequence of the regimen cycle can comprise one or more unit doses. As such the cosmetic method, within a given day, may comprise administering on one, or several occasions throughout the day, either the cosmetic composition comprising the biologically active compound or the placebo composition as appropriate depending upon the phase of the treatment cycle. Furthermore on each treatment occasion the user may be advised to administer one or more metered or unmetered doses of the appropriate composition.

In an even more highly preferred embodiment of the present invention a multi chambered pack is used such that throughout phase one of the treatment regimen the pack dispenses a mixture of a composition comprising a protease enzyme stabilised by high levels of polyhydric alcohol and a chassis oil in water emulsion comprising emollients and skin care active vitamin B₃ complex and that during phase two of the treatment regimen the pack dispenses the chassis oil in water emulsion alone.

Preparation of Compositions

The oil in water emulsions of the present invention are prepared by standard techniques well known to those skilled in the art. In general the aqueous phase and/or the oil phase would be prepared separately, with materials of similar phase partitioning being added in any order. The two phases will then be combined with vigorous stirring to form the emulsion. Any ingredients in the formulation with high volatility, or which are susceptible to hydrolysis at high temperatures, can be added post emulsification with gentle stirring.

### EXAMPLES

The following examples further illustrate the preferred embodiments within the scope of the present invention. These examples are given solely for the purpose of illustration and are not to be construed as limitations of the present invention as many variations of the invention are possible without departing from its spirit or scope. Unless otherwise indicated, all ingredients are expressed as a weight percentage of the active ingredient.

| | Example | Example | Example | Example | Example |
|---|---|---|---|---|---|
| | 1 | 2 | 3 | 4 | 5 |
| Ingredients | % w/w | % w/w | % w/w | % w/w | % w/w |
| DEIONISED WATER | qs | qs | qs | qs | qs |
| GLYCERIN | 11.07 | 6.94 | 5.87 | 10.00 | 12.00 |
| Niacinamide | 2.00 | 5.00 | 3.50 | 3.50 | 2.00 |
| Panthenol | 0.50 | 0.50 | 0.50 | 0.50 | 0.50 |
| Vitamin E Acetate | 0.50 | 0.50 | 0.50 | 0.50 | 0.50 |
| ISOHEXADECANE | 2.94 | 3.08 | 3.12 | 5.40 | 5.40 |
| POLYACRYLAMIDE & | 2.50 | 2.50 | 2.50 | 2.50 | 2.50 |
| C13-14 ISOPARAFFIN & | | | | | |
| LAURETH-7¹ | | | | | |
| DIMETHICONE & | 2.00 | 2.00 | 2.00 | 2.00 | 2.00 |
| DIMETHICONOL² | | | | | |
| ISOPROPYL | 1.33 | 1.33 | 1.33 | 2.40 | 2.40 |
| ISOSTEARATE | | | | | |
| SORBITAN STEARATE & | 1.00 | 1.00 | 1.00 | 1.00 | 1.00 |
| SUCROSE COCOATE³ | | | | | |
| CETEARYL GLUCOSIDE/ | — | — | — | 0.3 | 0.3 |
| CETEARYL ALCOHOL⁴ | | | | | |
| CETYL ALCOHOL | 0.71 | 0.74 | 0.75 | 0.73 | 0.73 |
| PETROLATUM | 0.00 | 3.00 | 0.00 | 5.00 | 0.00 |
| Isopropyl Palmitate | 0.50 | 0.50 | 0.50 | 0.00 | 0.00 |
| Behenyl Alcohol | 0.00 | 0.00 | 0.00 | 0.95 | 0.95 |
| SEFA COTTONATE | 0.66 | 0.69 | 0.70 | 1.20 | 1.20 |
| STEARYL ALCOHOL | 0.47 | 0.49 | 0.50 | 0.95 | 0.95 |
| BENZYL ALCOHOL | 0.25 | 0.25 | 0.25 | 0.25 | 0.25 |
| ETHYLPARABEN | 0.20 | 0.20 | 0.20 | 0.20 | 0.20 |
| PROPYLPARABEN | 0.10 | 0.10 | 0.10 | 0.10 | 0.10 |
| DISODIUM EDTA | 0.10 | 0.10 | 0.10 | 0.10 | 0.10 |
| POLYOXYETHYLENE- | 0.10 | 0.10 | 0.10 | 0.10 | 0.10 |
| 100 STEARATE³ | | | | | |
| STEARIC ACID | 0.10 | 0.10 | 0.10 | 0.10 | 0.10 |
| SODIUM HYDROXIDE | 0.04 | 0.05 | 0.05 | 0.05 | 0.05 |
| Protease enzyme⁵ | 0.01 | 0.01 | 0.01 | 0.01 | 0.01 |
| (Enzyme ppm) | 100.00 | 100.00 | 50.00 | 100.00 | 50.00 |

¹Supplied by Seppic, 75 Quai D' Orsay, Paris
²Supplied by Dow Corning, Kings Court, 185 Kinds Rd, Reading, Berks, RG1 4EX
³Supplied ICI, P0 Box 90, Wilton Centre, Middlesborough, Cleveland, England. TS6 8JE
⁴Supplied by Henkel, Dusseldorf, Germany
⁵Genencor International, Palo Alto, California, US
⁶Airspray International, Alkmaer, Netherlands

Manufacture of Lotion/Skin Cream

Heat a mixture of water, glycerine, Sorbitan stearate/sucrose cocoate (if applicable) and EDTA together using gentle stirring.

Simultaneously, mix and heat the following materials: Vitamin E Acetate, Isohexadecane, Isopropyl Isostearate, Cetyl Alcohol, Petrolatum, Ispropyl Palmitate, Behenyl Alcohol, Stearyl Alcohol, Ethyl Paraben, Propyl paraben, PEG 100 stearate, stearic acid and Cetearyl Glucoside/Cetearyl Alcohol (if applicable).

When the two mixtures are at 75-80C, add the mixtures together and mix at high shear to create an emulison.

Cool the mixture and add Niacinamide, panthenol, sodium hydroxide Polyacrylamide & C13-14 Isoparaffin & Laureth 7 during the cooling cycle.

At the point of finishing the batch add Dimethicone and Dimethiconol and mix briefly with high shear. Allow the mixture to cool to room temperature

Enzyme Solution

Mix the enzyme with a suitable solvent to dissolve the material, in the required amounts to achieve the final dilution.

Use of Final Pack (Symbio Pack)⁶

Assemble the package according to the suppliers' instructions and ensure the system is delivering both components before usage.

The compositions display excellent skin hydration, skin softness and skin smoothness benefits.

What is claimed is:

1 A composition in the form of an oil-in-water emulsion comprising:
(a) from about 0.0001% to about 1%, by weight, subtilisin protease enzyme;
(b) less than about 20%, by weight, polyhydric alcohol in the continuous phase of said emulsion; and
(c) from about 5% to about 25%, by weight, internal oil phase of said emulsion; and
(d) a salt selected from alkali metal and alkaline earth metal salts, and mixtures thereof, in an amount of from about 5 ppm to about 500 ppm based on the metal ion;
wherein said subtilisin protease enzyme exhibits its activity at pH greater than about 5.5; and
wherein the water activity of the external phase of said emulsion is greater than about 0.85.

2. A composition according to claim 1, comprising from about 0.001% to about 0.5%, of said subtilisin protease enzyme.

3. A composition according to claim 1, wherein said subtilisin protease enzyme is a bacterial serine protease enzyme obtained from bacteria selected from the group consisting of Bacillus amyloliquefaciens, Bacillus licheniformis and Bacillus subtilis.

4. A composition according to claim 1, wherein said subtilisin protease enzyme is a variant of a naturally occurring protease enzyme.

5. A composition according to claim 1, wherein said continuous phase comprises from about 0.5% to about 18%, of polyhydric alcohol.

6. A composition according to claim 5, wherein said continuous phase comprises from about 5% to about 12%, of polyhydric alcohol.

7. A composition according to claim 1, wherein the polyhydric alcohol is selected from the group consisting of glycerine; butylene glycol; propylene glycol; dipropylene glycol; polyethylene glycol; hexane triol; ethoxylated glycerine; propoxylated glycerine and mixtures thereof.

8. A composition according to claim 1, wherein the composition comprises from about 8% to about 15%, of internal oil phase.

9. A composition according to claim 1, wherein the salt is selected from sodium, calcium and magnesium salts, and mixtures thereof.

10. A composition according to claim 1, wherein the composition comprises from about 0.5% to about 15%, by weight, of a dermatologically acceptable emollient.

11. A composition according to claim 1, wherein the composition comprises from about 0.01% to about 20%, by weight, of a skin care active complex.

12. A composition according to claim 11, wherein the skin care active comprises a vitamin complex comprising vitamin B₃ compound or derivatives thereof; retinol compound or derivatives thereof; panthenol or derivatives thereof; and mixtures thereof.

13. A composition according to claim 1, wherein the protease enzyme exhibits its activity at a pH of from about 7.0 to about 9.0.

14. A composition according to claim 13, wherein the protease enzyme exhibits its activity at a pH of from about 8.0 to about 8.6.

15. A composition according to claim 1, wherein the external phase has a water activity of greater than 0.90.

16. A composition according to claim 15, wherein the external phase has a water activity of greater than 0.95.

17. A composition according to claim 1, wherein the composition is packaged in a pack comprising two or more chambers and a dispensing system:
(a) wherein the first chamber comprises an aqueous composition comprising:
(i) said subtilisin protease enzyme;
(ii) said polyhydric alcohol; and
(iii) less than about 20%, by weight, of water;
(b) wherein the second chamber comprises a placebo composition formulated as an oil-in-water emulsion; and
(c) wherein the compositions of the two chambers are dispensed from the dispensing system simultaneously.

18. A composition according to claim 17 wherein the weight ratio of the composition dispensed from the first chamber to the composition dispensed from the second, chamber is from about 1:1 to about 1:50.

19. A composition according to claim 18 wherein the weight ratio of the composition dispensed from the first chamber to the composition dispensed from the second chamber is from about 1:5 to about 1:30.

20. A composition according to claim 17 wherein the weight ratio of the composition dispensed from the first chamber to the composition dispensed from the second chamber is from about 1:15 to about 1:23.


SEQUENCE LISTING (1 sequence)

SEQ ID NO: 1 - protein, 277 aa, artificial
  amino acid sequence for BPN′ subtilisin protease
AQSVPYGVSQIKAPALHSQGYTGSNVKVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNKYGAYNGTSMASPHVAGAAALILSKHPNWTNTQVRSSLENTTTKLGDSFYYGKKGLINNVQAAAQ